(19) United States
(12) Patent Application Publication
(10) Pub. No.: US 2011/0021891 A1
(43) Pub. Date: Jan. 27, 2011

(54) NON-INVASIVE GLUCOSE MONITOR

(75) Inventors: Shingo YOKOYAMA, Hitachinaka (JP); Kyoichi TAKAHASHI, Fujisawa (JP); Hiromi SUGO, Kanegasaki (JP); Shoichi SATO, Ichinoseki (JP); Tadayuki ABE, Ichinoseki (JP)

Correspondence Address: MATTINGLY & MALUR, P.C., 1800 DIAGONAL ROAD, SUITE 370, ALEXANDRIA, VA, 22314, US

(73) Assignee: HITACHI MEDIA ELECTRONICS CO., LTD., Iwate (JP)

(21) Appl. No.: 12/841,555

(22) Filed: Jul. 22, 2010

(30) Foreign Application Priority Data
Jul. 23, 2009 (JP) 2009-171724

(51) Int. Cl. A61B 5/1455

(52) U.S. Cl. 600/316

(57) ABSTRACT

In a non-invasive glucose monitor is configured to, by using a heat conductive member having a body surface contacting part for contacting a surface of a human body at its one end, measures a first temperature adjacent to the body surface contacting part and a second temperature adjacent to the other end apart from the body surface. The computing device takes in data of the first and second temperature, environmental temperature, radiation heat from the body surface, reflection light caused by reflecting the light of the two different wavelength at the body surface contacting part. The computing device stores a relationship between parameters and blood glucose levels in advance, thereby converts the above-mentioned data to parameters, and calculates a blood glucose level by applying the parameters to the relationship.

### CLAIM OF PRIORITY

The present application claims priority from Japanese application serial No. 2009-171724, filed on Jul. 23, 2009, the contents of which are hereby incorporated by references into this application.

### FIELD OF THE INVENTION

The present invention relates to a non-invasive glucose monitor that measures a glucose level in blood in a non-invasive manner.

### BACKGROUND OF THE INVENTION

For a diabetic to do an insulin injection properly, it is necessary to measure a glucose level in blood day by day correctly.

As a method of measuring the blood glucose level, there is known an invasive method of taking a small amount of blood sample from a patient and measuring a density of glucose contained in the blood. As to such an invasive method, it since needs to take the blood sample from a patient, it not only gives a physical pain to the patient but also increases cost of consumables such as exsanguination use needles, and further accompanies a possible danger of infectious disease via the needles.

For this reason, a non-invasive glucose monitor has been proposed as one which is able not to give a physical pain to the patient as well as to eliminate the cost increase of consumables and such danger of infectious disease.

For example, JP-B-3767449 indicated as patent document 1 proposes a non-invasive glucose monitor of irradiating light with a predetermined wavelength to a living body and sensing the reflection light thereof thereby to measure a density of glucose in the blood.

### SUMMARY OF THE INVENTION

In order to enhance the measurement accuracy of the blood glucose level in comparison with the conventional art as disclosed in patent document 1, the present invention pays attention on a heat transfer rate from a body surface to outside thereof (a heat transfer rate per unit time) as a parameter relating to a blood flow rate.

Namely, the present invention uses a method in which, by contacting a member having a constant thermal resistance to a body surface, a temperature gradient caused within the member is measured to calculate a rate of heat transferring in the member, and the parameter relating to the blood flow rate is obtained from the heat transfer rate.

When adopting such a method to be used in the present invention, it since takes much time until an equilibrium temperature is reached, it is important to estimate the equilibrium temperature in a short measurement time.

The present invention is to provide a non-invasive glucose monitor capable of measuring the heat transfer rate from a human body in a short time to obtain a parameter relating to the blood flow rate and measuring the blood glucose level in high accuracy.

The present invention is basically constituted by as follows. It is comprised of:

a heat flux measurement device including a heat conductive member having a body surface contacting part for contacting a surface of a human body at its one end, a first temperature sensor with which the heat conductive member is provided adjacent to the body surface contacting part thereof, and a second temperature sensor with which the heat conductive member is provided adjacent to the other end apart from the body surface thereof;

an environmental temperature sensor that measures environmental temperature;

a radiation heat sensor that measures radiation heat from the body surface;

a light source that irradiates light having at least two different wavelengths toward the body surface contacting part;

a photo sensor that senses reflection light caused by reflecting the light at the body surface contacting part;

a computing device including a conversion part that converts respective outputs from the first temperature sensor, the second temperature sensor, the environmental temperature sensor, the radiation heat sensor and the photo sensor to respective parameters, and a processing part that stores a relationship between parameters and blood glucose levels and calculates a blood glucose level by applying the parameters converted from the respective outputs to the relationship, and a display part that displaying result outputted from the computing part.

For example, according to the present invention, after starting temperature measurement at the first temperature sensor and the second temperature sensor in the heat flux measuring device, a regression line is determined by making use of a least square method from temperature rise curves obtained at the first temperature sensor and the second temperature sensor with a predetermined time interval; relationships between the gradient of the regression line and equilibrium temperatures of the first temperature sensor and the second temperature sensor are respectively stored in advance as database; and the equilibrium temperature for a subject to be monitored is predicted from the database.

For example, a finger may be used as the body surface.

For example, preferably, the heat flux measuring device may be used while being cooled with a louver or a fan.

For example, the data base may be prepared in a form of grouping respectively with respect to items of at least room temperature, sex, age and anamnesis.

According to the present invention, it can provide a non-invasive monitor capable of measuring the heat transfer rate from a human body in short time to obtain a parameter relating to the blood flow rate and thereby measuring the blood glucose level in short time with high accuracy.

### BRIEF DESCRIPTION OF THE DRAWINGS

FIG. 1 is a thermal circuit showing a measurement principle of blood flow rate in the present invention's monitor.

FIG. 2 is an explanatory diagram illustrating relationships between measurement values by varieties of sensors and parameters derived therefrom.

FIG. 3 is a top view of a non-invasive glucose monitor of the present invention's embodiment.

FIG. 4 is a cross sectional view of a measurement part representing a first embodiment of the present invention.

FIG. 5 is a cross sectional view of a measurement part representing a second embodiment of the present invention.

FIG. 6 is a cross sectional view of a measurement part representing a third embodiment of the present invention.

FIG. 7 is temperature rise curves of subjects A and B.

FIG. 8 is a view showing a relationship between equilibrium temperature rise and gradient of regression line.

FIG. 9 is an example of database.

FIG. 10 is a cross sectional view of an optical sensor section.

FIG. 11 is a conceptual diagram showing data processing flow in the present monitor.

FIG. 12 is a plotting diagram of calculated values of glucose density according to the present invention and measured values of glucose density according to enzyme electrode method.

FIG. 13 is a diagram showing a manipulation sequence of the present monitor.

### EMBODIMENTS OF THE INVENTION

Herein below, embodiments of the present invention will be explained with reference to drawings.

### Embodiment 1

At first, an amount of heat dissipated from a human body will be considered. Convective heat transfer that is a principal cause of the amount of heat dissipation relates to a temperature difference between the environment temperature (room temperature) and the temperature of body surface, and an amount of heat dissipation due to radiation that is another cause is proportional to the fourth power of absolute temperature of the body surface according to Stefan-Boltzmann law. Accordingly, it is understood that the amount of heat dissipation from a human body is related to the room temperature and the temperature of body surface.

On the other hand, an amount of oxygen supply that is a cause relating to an amount of heat generation is expressed by a product of hemoglobin density, oxygen saturation of hemoglobin and a blood flow rate.

Hemoglobin density can be measured from an absorbance at a wavelength (equal absorption wavelength) where mol absorption coefficients of oxygen coupling type hemoglobin and reduced (deoxidized) type hemoglobin become equal to each other.

The oxygen saturation of hemoglobin can be determined by measuring the above-mentioned absorbance at the equal absorption wavelength and at least another absorbance at the other wavelength in which ratio of mol absorption coefficients of oxygen coupling type hemoglobin and reduced (deoxidized) type hemoglobin is already known and by resolving simultaneous equations using the two wavelength (the equal absorption wavelength and the other wavelength).

Namely, the hemoglobin density and the oxygen saturation of hemoglobin can be obtained by measuring absorbances of at least two wavelengths. And remaining one is a flow rate of blood.

Although the blood flow rate can be measured with varieties of methods, an example of such measurement methods will be explained with reference to a drawing.

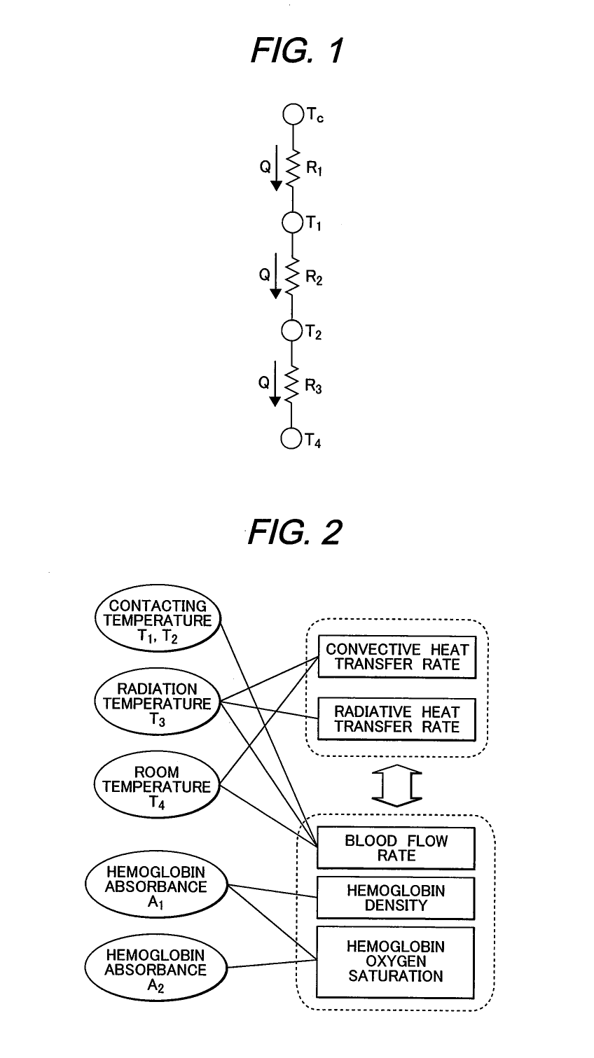

FIG. 1 is a thermal circuit showing a measurement principle of blood flow rate in the present invention's monitor.

In FIG. 1, a human body is performing heat exchange with the external of the human body so as to maintain its temperature at a deep part of the human body at constant 37° C. Therefore, by measuring the heat transfer rate Q transferring between temperature T_{c} at the deep part of the human body and temperature T₁ at the body surface, the thermal resistance R₁ between T_{C} and T₁ can be determined. Since this thermal resistance R₁, namely, thermal conductivity in human body tissue has a correlation with the blood flow rate, the blood flow rate can be estimated from the thermal resistance R₁. Measurement of the heat transfer rate Q may be done by preparing a block having a certain constant heat resistance R₂ and measuring the temperatures (T₁ and T₂) at both ends of the block whose one end contacts the body surface (Q=R₂/(T₁-T₂)). T₁ is of temperature at one end side where the block contacts the body surface, and T₂ is of temperature at the other end side opposite to the one end side contacting the body surface. The heat passing through the block from T₁ is dissipated from T₂ to the room temperature T₄ via the heat resistance R₃.

R₁ is determined according to the following formula.

[MATHEMATICAL FORMULA 1]

R₁=(T_{C}−T₁)R₂/(T₁−T₂) (1)

Herein, T_{C} is a constant temperature at 37° C. as referred to above, therefore, when measuring T₁ and T₂ while fixing R₂, R₁ is determined, and the blood flow rate having correlation with R₁ can be estimated.

Further, when also measuring the body surface temperature T₃ with a radiation temperature meter, an amount of transferred radiation heat from the body surface can be estimated. A parameter suggesting the blood flow rate estimated from R₁ is defined as X₅.

According to our knowledge, we found that the measurement data necessary for determining the glucose density in blood with a mathematical model (described later of the present embodiments) is of room temperature (environmental temperature), temperature gradient inside the block contacting to the body surface, temperature due to radiation from the body surface and absorbances of at least two wavelengths.

FIG. 2 is an explanatory diagram illustrating relationships between measurement values by varieties of sensors in the present monitor and parameters derived therefrom.

In FIG. 2, the block to be contacted to the body surface as explained in FIG. 1 is prepared, and the two kinds of temperatures T₁ and T₂ are measured with two temperature sensors disposed at the two portions. The radiation temperature T₃ of the body surface and the room temperature T₄ are measured separately. Further, absorbances A₁ and A₂ are measured with at least two kinds of wavelengths relating to hemoglobin absorption. A parameter relating to “blood flow rate” is obtained from the temperatures T₁˜T₄, a parameter relating to “amount of transferred radiation heat” is obtained from the temperature T₃ and parameters relating to “amount of transferred convective heat” are respectively obtained from the temperature T₃ and the temperature T₄. Further, a parameter relating to “hemoglobin density” is obtained from the absorbance A₁, and a parameter relating to “oxygen saturation of hemoglobin” is obtained from the absorbances A₁ and A₂.

FIG. 3 is a top view of a non-invasive glucose monitor provided with an embodiment of the present invention.

In FIG. 3, on the top face of a non-invasive glucose monitor 100, a manipulating portion 11, a measurement part 12 on which a finger serving as a measurement object is placed, and a display part 13 that displays such as the measurement result, the state of the monitor and measurement values are provided. At the manipulating portion 11, four push buttons 11a˜11d are disposed for performing manipulation of items on the top face of the non-invasive glucose monitor 100. A cover 14 is provided for the measurement part 12, and when the cover 14 is opened (the drawing shows the state when the cover is opened), a finger placing part 15 having an elliptical shape circumference appears. In the finger placing part 15, an opening end 16a for a radiation temperature sensor part 16, a heat flux sensor part 17 and an optical sensor part 18 are provided.

In the non-invasive glucose monitor 100 according to the present embodiment, although the thick skin of a finger top is used as the body surface, other body surfaces can also be used.

In order to determine the thermal resistance R₁, the equilibrium temperatures T₁ and T₂ are necessitated, however, in an actual measurement the following problems arise.

(1) When an amount of leakage heat transfer rate flow transferring from the finger to portions other than the block is large, a measurement error increases.

(2) It takes time until an equilibrium temperature for performing measurement is reached.

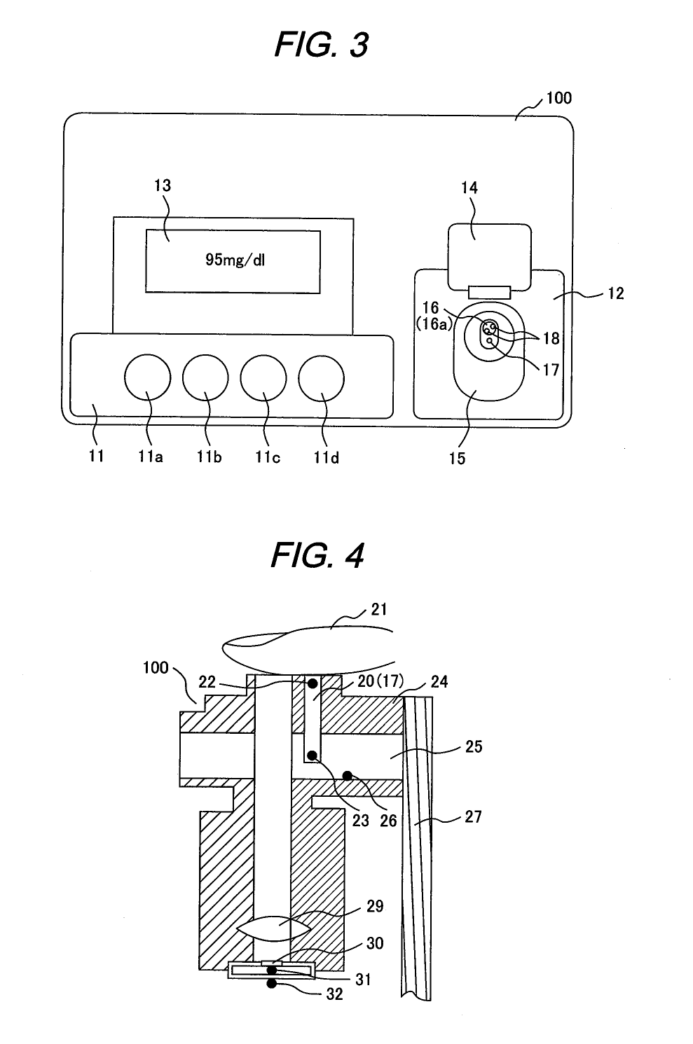

The present embodiment has a structure for solving such problems as follows. FIG. 4 is a cross sectional view showing details of the measurement part 12 (the radiation temperature sensor part 16 and the optical sensor parts 18 of FIG. 3 are omitted).

In FIG. 4, the heat flux sensor part 17 as shown in FIG. 3 is constituted by a heat conductive member 20, a thermister 22 (T₁) disposed adjacent to the part where a finger 21 contacts to an one end of the heat conductive member 20 and another thermister 23 (T₂) disposed at the other end (bottom end) opposite to the one end of the heat conductive member 20. It is desirable to constitute an enclosure 24 around the heat conductive member 20 in a heat insulating structure (for example, either the thermal conductivity of the enclosure 24 is set lower than the thermal conductivity of the heat conductive member 20 or the enclosure 24 is constituted in a vacuum heat insulated structure) so as to prevent the heat from leaking largely from the finger 21 serving as the measurement part to the enclosure 24 via the heat conductive member 20.

A hollow part 25 is of a space for naturally air cooing the heat conductive member 20 to a room temperature via an outer frame 27 after finishing a body temperature measurement at the heat flux sensor part 17, and the end side of the heat conductive member 20 opposite to the finger contacting part is positioned within the hollow part 25. The thermister 26 is provided at the hollow part 25 to measure the room temperature. Further, an infrared lens 29 for optical measurement is disposed at a position inside the monitor 100 where the thick of the finger can be seen. A pyroelectricity sensor 31 is arranged below the infrared lens 29 via an infrared transmission window 30. Further, still another thermister 32 is disposed near the pyroelectricity sensor 31.

This infrared lens 29 is for focusing electromagnetic waves from the finger 21. The surface temperature of the finger 21 is sensed by the thermister 32 disposed near the pyroelectricity sensor 31 to which the electromagnetic waves are collected.

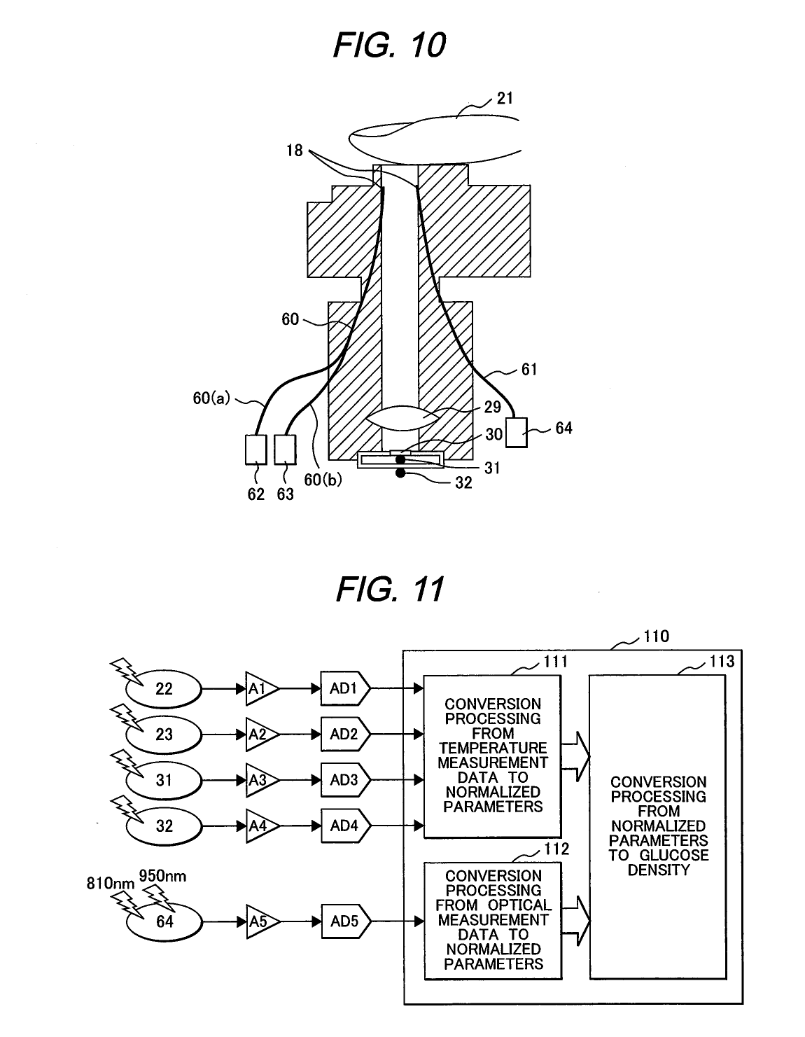

This optical measurement will be explained in connection with FIG. 10 later.

### Embodiment 2

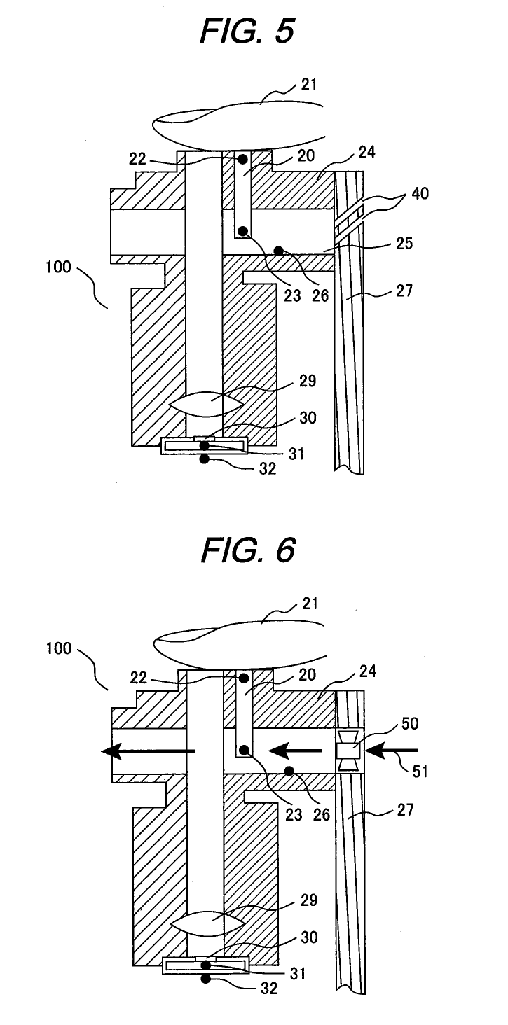

FIG. 5 is a cross sectional view showing details of a measurement part of the monitor 100 provided with another embodiment.

FIG. 5 shows an instance where the outer frame 27 is provided with louvers 40 for cooling acceleration of the hollow part 25. With this measure, cooling of the hollow part 25 is accelerated and the time for cooling the same to the room temperature is shortened.

### Embodiment 3

FIG. 6 is a cross sectional view showing details of a measurement portion of the monitor 100 provided with still another embodiment.

FIG. 6 shows an instance where the outer frame 27 is provided with a fan 50 for further cooling acceleration of the hollow part 25. With this measure, cooling of the hollow part 25 is further accelerated by cooling wind 51 and the time for cooling the same to the room temperature is greatly shortened.

Now, a method of predicting the equilibrium temperature will be explained.

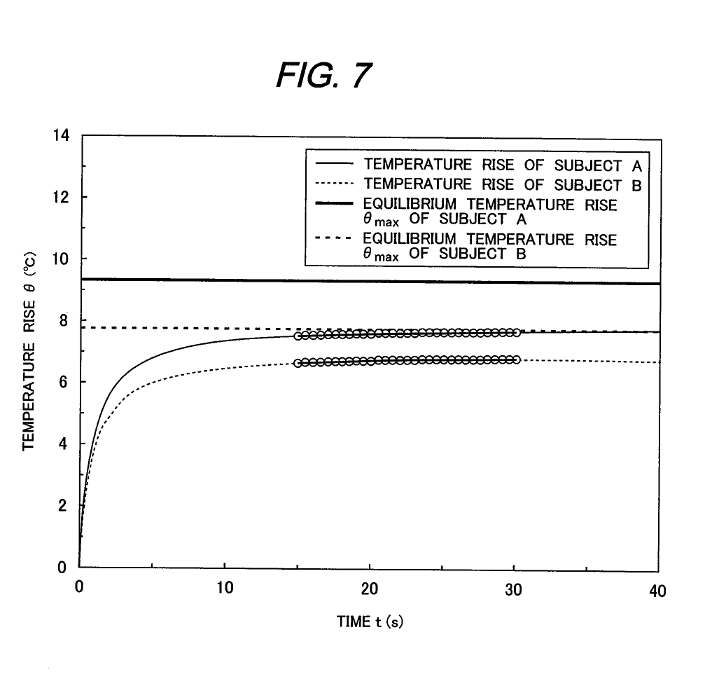

FIG. 7 is a diagram showing temperature rising values (herein below will be called as temperature rise) from the initial of subjects A and B (both males with slight age difference) measured with the thermister 22.

In FIG. 7, a forefinger in right side is used as the measurement portion. In the drawing, the ordinate shows temperature rise θ, the abscissa shows time t, a fine solid line shows temperature rise of subject A, a fine dotted line shows temperature rise of subject B, a bold solid line shows an equilibrium temperature rise θₘₐₓ of subject A and a bold dotted line shows an equilibrium temperature rise θₘₐₓ of subject B respectively. Herein, θ and θₘₐₓ are expressed by the following formulas.

[MATHEMATICAL FORMULA 2]

θ≡T−Tᵢ (2)

[MATHEMATICAL FORMULA 3]

θₘₐₓ=Tₘₐₓ−Tᵢ (3)

Herein, T is temperature in (° C.), suffix i: initial condition and suffix max: equilibrium condition

When classifying features of the temperature rise curves after contacting the finger to the temperature sensor with a constant pushing pressure, the initial portion thereof is considered that is affected by such as the surface temperature of the finger and the initial temperature of the temperature sensor, and reflects the heat transfer from the finger to the temperature sensor. On the other hand, after a certain time has passed, the portion thereof is considered to reflect heat transferred from the finger deep part to the surface due to biological reaction at the finger deep part. Accordingly, the present invention excluded the initial portion that contains large error, noted to a section obtained after a certain time has passed from the initial portion that contains a limited error and devised to estimate the equilibrium temperature by making use of the temperature rise curve of this section.

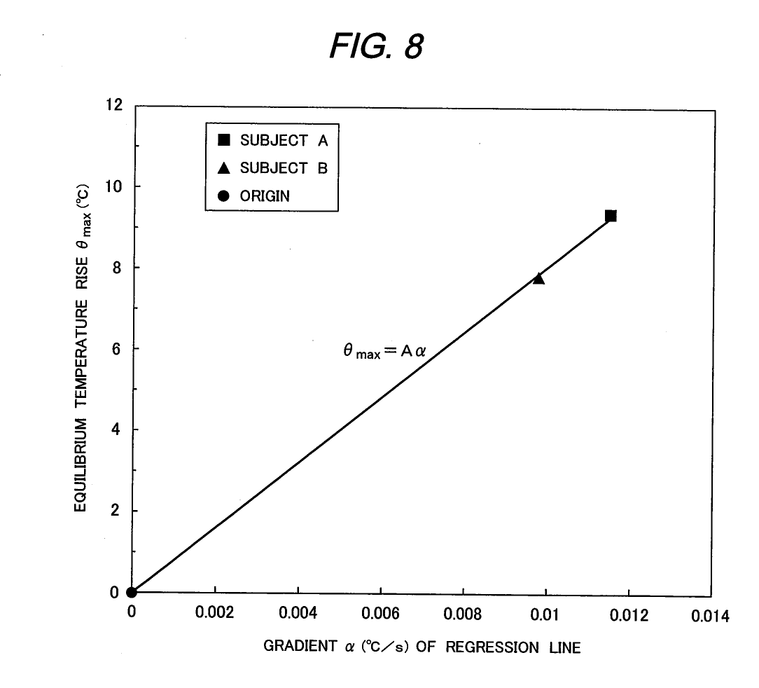

When explaining more specifically, in FIG. 7, at first the initial portion of t=0˜15s is excluded, by making use of data (illustrated by circles) in the section of t=15˜30s (section adjacent to a terminal portion of the temperature rise), regression lines (overwritten lines on the circles) are determined through least square method, and the gradient a thereof is calculated. Then, as shown in FIG. 8, while plotting the equilibrium temperature rise θₘₐₓ for the ordinate and the gradient a of the regression lines for the abscissa, a straight line passing through the origin (when α=0, θₘₐₓ=0) is drawn to determine the gradient A. Herein, α and A are expressed by the following formulas.

[MATHEMATICAL FORMULA 4]

θ=αt+β (4)

[MATHEMATICAL FORMULA 5]

θₘₐₓ=Aα (5)

β in the formula (4) is an intercept (which is not used in the prediction in the present invention) of the regression line. In FIG. 8, by making use of three points of similar subjects A (illustrated by a solid square) and B (illustrated by a sold triangle) and the origin (illustrated by a solid circle), a straight line is drawn. Although it is confirmed that both data of the subjects A and B are sit on the straight line, because the origin is used, it is indicated that the gradient A is determined unambiguously if at least a single data of a similar subject is obtained.

When a body temperature of another subject similar to the subjects A and B is measured at the same room temperature (an initial temperature), a correct equilibrium temperature Tₘₐₓ is predicted in a measurement time of about 40s by making use of the formula (5) determined according to FIG. 8. Namely, according to the present invention, the temperature rise curves and the equilibrium temperatures in varieties of initial conditions are determined in advance experimentally and are stored as database, and an equilibrium temperature for a similar subject is predicted in a short time as well as in high accuracy, in other words, the maintenance of such database becomes important.

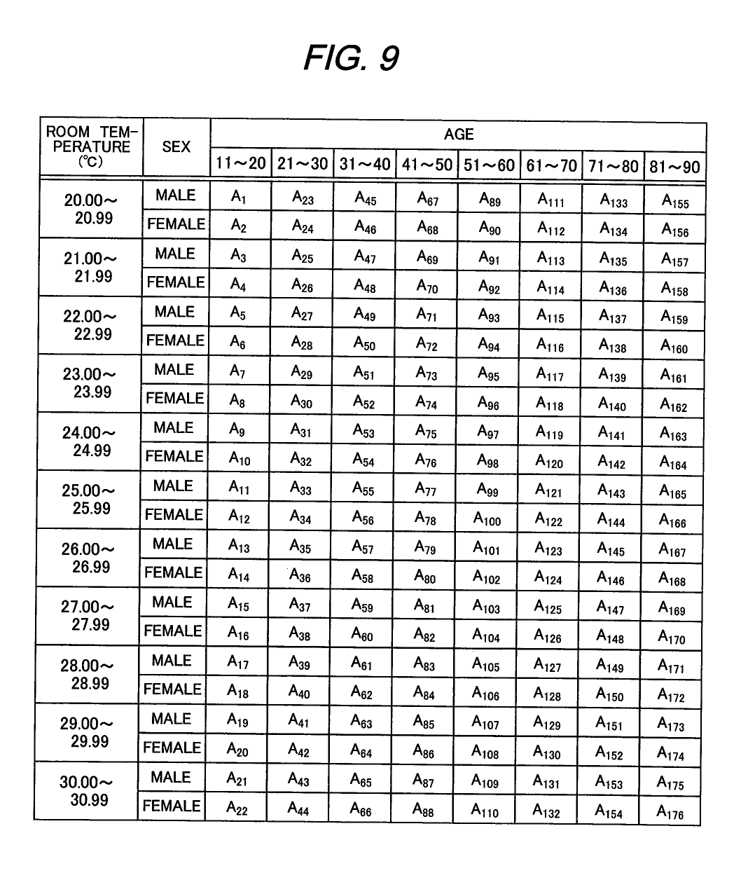

An example of such database is shown in FIG. 9. This database organizes the gradients A in the formula (5) according to room temperatures, sex and ages (in FIG. 9 176 sorts of A₁˜A₁₇₆ are shown). In FIG. 9, although the gradients A are organized by sectioning the room temperature of 20˜30° C. in 0.99° C. segment and ages of 11˜90 in 10 segment depending on sex, the database can be prepared by changing such as the range of the items and the segmentation depending on the necessity and by adding conditions such as anamnesis. Further, the method of predicting the equilibrium temperature as explained above can be applied likely to the thermister 23, and R₁ having correlation with the blood flow rate can be estimated finally according to the formula (1).

FIG. 10 is a diagram showing an exemplary constitution for performing two wavelengths measurement with two light sources 62 and 63 and a single sensor 64.

With reference to FIG. 10, when explaining the optical measurement by the optical sensor portion 18 as shown in FIG. 3, the optical sensor portion 18 is for measuring hemoglobin density and hemoglobin oxygen saturation that are necessary for determining the amount of oxygen supply. In order to measure the hemoglobin density and hemoglobin oxygen saturation, the absorbance measurement with at least two wavelengths is necessitated. FIG. 10 shows an exemplary constitution for performing the two wavelengths measurement with two light sources 62 and 63 and a single sensor 64.

At the optical sensor portion 18, end portions of two optical fibers 60 and 61 are located. The optical fiber 60 is a light irradiation use optical fiber, and the optical fiber 61 is a light receiving use optical fiber. The optical fiber 60 is connected to branching optical fibers 60a and 60b. Further, the other end terminals of these fiber 60a and 60b are connected to the light sources 62 and 63 for the two wavelengths respectively. To the end terminal of the light receiving use optical fiber 61, the sensor 64 is arranged. The light source 62 emits light having wavelength of 810 nm, and the light emitting diode 63 emits light having wavelength of 950 nm. The wavelength of 810 nm is the equal absorption wavelength where the mol absorption coefficients of oxygen coupling type hemoglobin and reduced (deoxidized) type hemoglobin become equal, and the wavelength of 950 nm is a wavelength where the difference of the mol absorption coefficients of oxygen coupling type hemoglobin and reduced type hemoglobin becomes large.

The two light sources 62 and 63 emit light in time sharing, and the light emitted from the light sources 62 and 63 is irradiated to the finger 21 of the subject via the light irradiating use optical fiber 60. The light irradiated to the finger 21 is reflected by the skin of the finger 21, makes incident to the light receiving use optical fiber 61 and is sensed by the sensor 64. When the light irradiated to the finger is reflected by the skin of the finger, a part of the irradiated light enters into tissues through the skin and is absorbed by hemoglobin in blood flowing through capillaries. The measurement data by the sensor 64 is reflectance R and the absorbance is calculated approximately with log(1/R). Radiation is performed respectively with light having wavelengths 810 nm and 950 nm, respective reflectance Rs are measured and respective log(1/R)s are determined, thereby, absorbance A₁ for wavelength 810 nm and absorbance A₂ for wavelength 950 nm are obtained.

When assuming reduced type hemoglobin density as [Hb] and oxygen coupling type hemoglobin density as [HbO₂], the absorbance A₁ and absorbance A₂ are expressed by the following formulas.

$$\begin{matrix}[ MATHEMATICAL FORMULA 6 ] & \\ \begin{matrix}{A}_{1} = a \times ( \begin{matrix}[ Hb ] \times {A}_{Hb} ( 810 nm ) + \\ [ {Hb O}_{2} ] \times {A}_{HbO 2} ( 810 nm )\end{matrix} ) \\ = a \times ( [ Hb ] + [ {Hb O}_{2} ] ) \times {A}_{HbO 2} ( 810 nm )\end{matrix} \\ \begin{matrix}{A}_{2} = a \times ( [ Hb ] \times {A}_{Hb} ( 950 nm ) + [ {Hb O}_{2} ] \times {A}_{HbO 2} ( 950 nm ) ) \\ = a \times ( [ Hb ] + [ {Hb O}_{2} ] ) \times \{ \begin{matrix}\begin{matrix}[ 1 - [ {Hb O}_{2} ] / ( [ Hb ] + [ {Hb O}_{2} ) ] \times \\ {A}_{Hb} ( 950 nm ) + [ {Hb O}_{2} ] /\end{matrix} \\ ( [ Hb ] + [ {Hb O}_{2} ] ) \times {A}_{HbO 2} ( 950 nm )\end{matrix} \}\end{matrix} & ( 6 )\end{matrix}$$

A_{Hb}(810 nm), A_{HbO2}(810 nm), A_{Hb}(950 nm) and A_{HbO2}(950 nm) are mol absorption coefficients at the respective wavelengths of reduced type hemoglobin and oxygen coupling type hemoglobin that are already known. “a” is a proportional coefficient. Hemoglobin density [Hb]+[HbO₂] and hemoglobin oxygen saturation [HbO₂]/([Hb]+[HbO₂]) are determined as follows from the above formulas.

[MATHEMATICAL FORMULA 7]

[Hb]+[HbO₂]=A₁/a×A_{HbO2}(810 nm)

[HbO₂]/([Hb]+[HbO₂])=(A₂×A_{HbO2}(810 nm)−A₁×A_{Hb}(950 nm))/A₁×(A_{HbO2}(950 nm)−A_{Hb}(950 nm)) (7)

Herein, although an example where the hemoglobin density and hemoglobin oxygen saturation are measured through measurement of absorbances at two wavelengths, it is possible to reduce influences due to disturbing components and to enhance the measurement accuracy through measurement of the absorbances at not less than three wavelengths.

FIG. 11 is a conceptual diagram showing data processing flow in the present monitor.

As seen from FIG. 11, in the monitor of the present embodiment, five sensors consisted by the thermister 22, thermister 23, pyroelectricity sensor 31, thermister 32 and sensor 64 are included. Since the sensor 64 measures absorbances at the wavelengths of 810 nm and 950 nm, six kinds of measurement values are inputted to the monitor.

Five kinds of analog signals are digital converted by analog/digital converters AD₁˜AD₅ via respective amplifiers A₁˜A₅. From the digital converted values, a conversion parts 111 and 112 calculate normalized parameters xᵢ (i=1, 2, 3, 4, 5) in a computing device 110. xᵢ can be expressed specifically in the following manner (a₁˜a₅ are proportional coefficients).

[MATHEMATICAL FORMULA 8]

Parameter proportional to radiation heat;

x₁=a₁×(T₃)⁴

Parameter proportional to heat convection;

x₂=a₂×(T₄−T₃)

Parameter proportional to hemoglobin density;

x₃=a₃×(A₁/a×A_{HbO2}(810 nm))

Parameter proportional to hemoglobin oxygen saturation;

x₄=a₄×(A₂×A_{HbO2}(810 nm)−A₁×A_{Hb}(950 nm))/(A₁×A_{HbO2}(950 nm)−A_{Hb}(950 nm))

Parameter proportional to blood flow rate;

x₅=a₅/R₁ (8)

Subsequently, normalized parameters Xᵢ are calculated from an average value and a standard deviation of the parameters xᵢ obtained from data of many actual healthy persons and diabetics. The normalized parameters Xᵢ (i=1, 2, 3, 4, 5) are calculated from respective parameters xᵢ according to the following formula.

[MATHEMATICAL FORMULA 9]

Xᵢ=(xᵢ×xₐ)/SD(xᵢ) (9)

xᵢ: parameters

xₐ: average value of parameters xᵢ

SD(xᵢ): standard deviation of parameters

With the above mentioned five normalized parameters Xᵢ, a conversion calculation to the glucose density to be finally displayed is performed with a processing part 113. Programs necessary for the processing calculation are stored in a ROM built-in in a microprocessor incorporated in the monitor 100. Further, a memory region necessary for the processing calculation is likely secured in a RAM incorporated in the monitor. Results of the processing calculation are displayed on a liquid crystal display portion.

In the ROM, as program constitutional elements necessary for the processing calculation, particularly, a function for determining the glucose density C is stored. The function is defined in the following manner. At first, C is expressed by the following formula (10). aᵢ (i=0, 1, 2, 3, 4, 5) are determined in advance from a plurality of measurement data. Steps of determining coefficient “aᵢ” are as follows.

(1) To prepare multiple regression formula indicating a relationship between normalized parameters and glucose density C.

(2) To determine a normal equation (simultaneous equation) relating to the normalized parameters from a formula obtained through least square method.

(3) To determine values of aᵢ (i=0, 1, 2, 3, 4, 5) from the normal equation and substitute the same in the multiple regression formula.

At first, the following regression formula (10) indicating a relationship between glucose density C and normalized parameters X₁, X₂, X₃, X₄, and X₅.

$$\begin{matrix}[ MATHEMATICAL FORMULA 10 ] & \\ C = f ( {X}_{1} , {X}_{2} , {X}_{3} , {X}_{4} , {X}_{5} ) \\ = {a}_{0} + {a}_{1} {X}_{1} + {a}_{2} {X}_{2} + {a}_{3} {X}_{3} + {a}_{4} {X}_{4} + {a}_{5} {X}_{5} & ( 10 )\end{matrix}$$

Subsequently, in order to determine a multiple regression formula that minimizes an error from the glucose density measurement values through enzyme electrode method, least square method is used. When assuming the residual sum of squares as D, D is expressed by the following formula.

$$\begin{matrix}[ MATHEMATICAL FORMULA 11 ] & \\ D = \sum {d}_{i}^{2} ( i = 1 \sim n ) \\ = \sum {( {C}_{i} - f ( {X}_{i 1} , {X}_{i 2} , {X}_{i 3} , {X}_{i 4} , {X}_{i 5} ) )}^{2} \\ = \sum {( {C}_{i} - ( \begin{matrix}{a}_{0} + {a}_{1} {X}_{i 1} + {a}_{2} {X}_{i 2} + \\ {a}_{3} {X}_{i 3} + {a}_{4} {X}_{i 4} + {a}_{5} {X}_{i 5}\end{matrix} ) )}^{2} & ( 11 )\end{matrix}$$

Since the residual sum of squares D will minimize when the formula (11) is partially differentiated by a₀, a₁, . . . a₅ and the same assume zero, the following formulas are obtained.

[MATHEMATICAL FORMULA 12]

∂D/∂a₀=−2Σ(Cᵢ−(a₀+a₁Xᵢ₁+a₂Xᵢ₂+a₃Xᵢ₃+a₄Xᵢ₄+a₅Xᵢ₅))=0

∂D/∂a₁=−2ΣXᵢ₁(Cᵢ−(a₀+a₁Xᵢ₁+a₂Xᵢ₂+a₃Xᵢ₃+a₄Xᵢ₄+a₅Xᵢ₅))=0

∂D/∂a₂=−2ΣXᵢ₂(Cᵢ−(a₀+a₁Xᵢ₁+a₂Xᵢ₂+a₃Xᵢ₃+a₄Xᵢ₄+a₅Xᵢ₅))=0

∂D/∂a₃=−2ΣXᵢ₃(Cᵢ−(a₀+a₁Xᵢ₁+a₂Xᵢ₂+a₃Xᵢ₃+a₄Xᵢ₄+a₅Xᵢ₅))=0

∂D/∂a₄=−2ΣXᵢ₄(Cᵢ−(a₀+a₁Xᵢ₁+a₂Xᵢ₂+a₃Xᵢ₃+a₄Xᵢ₄+a₅Xᵢ₅))=0

∂D/∂a₅=−2ΣXᵢ₅(Cᵢ−(a₀+a₁Xᵢ₁+a₂Xᵢ₂+a₃Xᵢ₃+a₄Xᵢ₄+a₅Xᵢ₅))=0

(i=1˜n) (12)

When assuming that the average values of C and X₁˜X₅ are C_{mwan} and X₁ₘₑₐₙ˜X₅ₘₑₐₙ, since Xᵢₘₑₐₙ becomes zero (i=1˜5), the following formula (13) is obtained from formula (10).

$$\begin{matrix}[ MATHEMATICAL FORMULA 13 ] & \\ {a}_{0} = {C}_{mean} - {a}_{1} {X}_{1 mean} - {a}_{2} {X}_{2 mean} - {a}_{3} {X}_{3 mean} - {a}_{4} {X}_{5 mean} \\ = {C}_{mean} & ( 13 )\end{matrix}$$

Further, variation/covariation between the normalized parameters is expressed by the following formula (14), and covariation of the normalized parameters Xᵢ (i=1˜5) and C is expressed by the following formula (15).

[MATHEMATICAL FORMULA 14]

Sᵢⱼ=Σ(Xₖᵢ−Xᵢₘₑₐₙ)(Xₖⱼ−Xⱼₘₑₐₙ)=ΣXₖᵢXₖⱼ(i,j=1˜5,k=1˜n) (14)

[MATHEMATICAL FORMULA 15]

S_{iC}=Σ(Xₖᵢ−Xᵢₘₑₐₙ)(Cₖ−Cₘₑₐₙ)=ΣXₖᵢ(Cₖ−Cₘₑₐₙ)(i,j=1˜5,k=1˜n) (15)

When substituting the formulas (13), (14), (15) into formula (12) and organizing the same, the following simultaneous equation (normal equations) is obtained, and when solving the same, a₁˜a₅ are obtained.

[MATHEMATICAL FORMULA 16]

a₁S₁₁+a₂S₁₂+a₃S₁₃+a₄S₁₄+a₅S₁₅=S_{1C}

a₁S₂₁+a₂S₂₂+a₃S₂₃+a₄S₂₄+a₅S₂₅=S_{2C}

a₁S₃₁+a₂S₃₂+a₃S₃₃+a₄S₃₄+a₅S₃₅=S_{3C}

a₁S₄₁+a₂S₄₂+a₃S₄₃+a₄S₄₄+a₅S₄₅=S_{4C}

a₁S₅₁+a₂S₅₂+a₃S₅₃+a₄S₅₄+a₅S₅₅=S_{5C} (16)

The constant term a₀ is obtained by making use of the formula (13). aᵢ (i=0, 1, 2, 3, 4, 5) as obtained as in the above manner are stored in the ROM at the time when the monitor is manufactured. During actual measurement by the monitor, the glucose density C is calculated by substituting normalized parameters X₁˜X₅ determined from the measurement values into the regression formula (10).

Subsequently, a specific example of calculation process of the glucose density will be shown. From many data measured in advance in connection with healthy persons and diabetics, the coefficients in formula (10) have been determined, and the following calculation formula of the glucose density is stored in the ROM in the microprocessor.

[MATHEMATICAL FORMULA 17]

C=105.0−20.0X₁+38.5X₂−78.9X₃−15.2X₄−41.1X₅ (17)

X₁˜X₅ are ones obtained by normalizing the parameters x₁˜x₅. When assuming that the distribution of the parameters is a normal distribution, 95% of the normalized parameters takes values between −2˜+2.

As an example of measurement values of a healthy person, when substituting normalized parameters X₁=+0.10, X₂=−0.02, X₃=+0.04, X₄=−0.20, and X₅=+0.20 into the above formula, C=94 mg/dl is obtained.

Further, as an example of measurement values of a diabetic, when substituting normalized parameters X₁=−1.10, X₂=+0.10, X₃=−0.84, X₄=−1.04, and X₅=−0.20 into the above formula, C=221 mg/dl is obtained.

A measurement result according to an enzyme electrode method that is a conventional measurement method in which blood obtained by taking a blood sample is reacted with a reagent and an amount of blood glucose is measured by measuring amount of electrons generated by the reaction and a measurement result according to one embodiment of the present invention will be explained herein below. As an example of measurement values of a healthy person, at the time when the glucose density is determined as 89 mg/dl according to the enzyme electrode method, when the normalized parameters X₁=+0.10, X₂=−0.02, X₃=+0.04, X₄=−0.20, and X₅=+0.20 that are obtained at the same time through measurement according to the present method are substituted into the above formula, C=94 mg/dl is obtained. Further, as an example of measurement values of a diabetic, at the time when the glucose density is determined as 238 mg/dl according to enzyme electrode method, when the normalized parameters X₁=−1.10, X₂=+0.10, X₃=−0.84, X₄=−1.04, and X₅=−0.20 that are obtained at the same time through measurement according to the present method are substituted into the above formula, C=221 mg/dl is obtained. From the above results, it was confirmed that the glucose density is determined in high accuracy with the method according to the present invention.

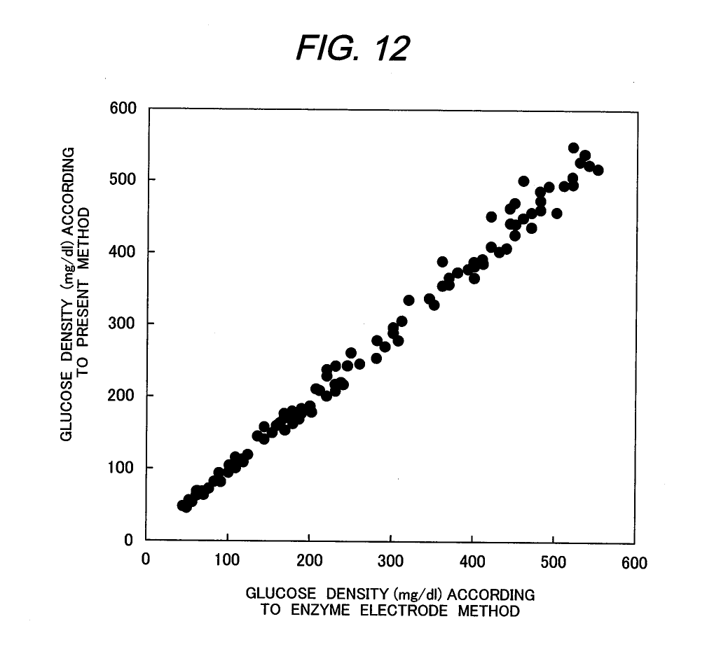

FIG. 12 is a diagram obtained by plotting measurement values of a plurality of patients in which the ordinate is calculation values of glucose density according to the present method and the abscissa is the measurement values of glucose density according to enzyme electrode method. Through measurement of the amount of oxygen supply and the blood flow rate according to the present method, a good correlation is obtained.

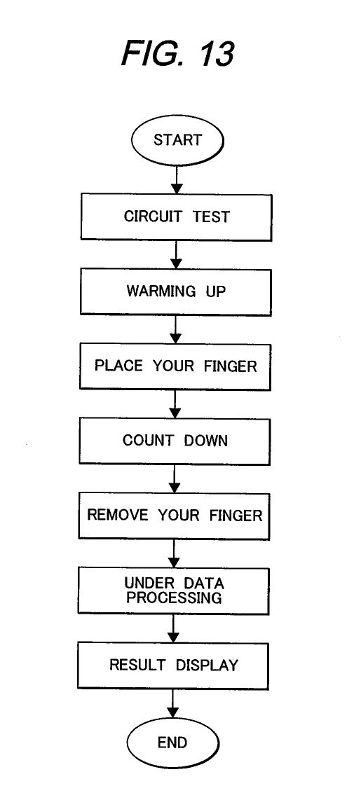

FIG. 13 shows a manipulation sequence of the present monitor.

In FIG. 13, when turning on the power source by pushing a button in the manipulation portion of the monitor, “warming up” is displayed on the liquid crystal display, and electronic circuits in the monitor are warmed up. At the same time, a checking program is operated to automatically check the electronic circuits. When the “warming up” is completed, “place your finger” is displayed on the liquid crystal display. When the finger is placed on the finger placing portion, a count down is displayed on the liquid crystal display. When the count down is completed, “remove your finger” is displayed on the liquid crystal display. When the finger is removed from the finger placing portion, “under data processing” is displayed on the liquid crystal display. Thereafter, an amount of blood glucose is displayed on the liquid crystal display.

At this moment, the displayed amount of blood glucose is stored in an IC card together with date and time. When the displayed amount of blood glucose is read out, a button in the manipulation portion is pushed. After about one minute, the monitor is rendered to a condition of waiting the subsequent measurement in which “place your finger” is displayed on the liquid crystal display.

As has been explained above, according to the present invention, it can be predicted in short time as well as in high accuracy.

What is claimed is:

1. A non-invasive glucose monitor comprising:
a heat flux measurement device including a heat conductive member having a body surface contacting part for contacting a surface of a human body at its one end, a first temperature sensor with which the heat conductive member is provided adjacent to the body surface contacting part thereof, and a second temperature sensor with which the heat conductive member is provided adjacent to the other end apart from the body surface thereof;
an environmental temperature sensor that measures environmental temperature;
a radiation heat sensor that measures radiation heat from the body surface;
a light source that irradiates light having at least two different wavelengths toward the body surface contacting part;
a photo sensor that senses reflection light caused by reflecting the light at the body surface contacting part;
a computing device including a conversion part that converts respective outputs from the first temperature sensor, the second temperature sensor, the environmental temperature sensor, the radiation heat sensor and the photo sensor to respective parameters, and a processing part that stores a relationship between parameters and blood glucose levels and calculates a blood glucose level by applying the parameters converted from the respective outputs to the relationship, and a display part that displaying result outputted from the computing part.

2. The non-invasive glucose monitor according to claim 1,
wherein the computing device is configured to, after starting temperature measurement at the first temperature sensor and the second temperature sensor in the heat flux measuring device, determines a regression line by making use of a least square method from temperature rise curves obtained at the first temperature sensor and the second temperature sensor with a predetermined time interval, stores relationships between the gradient of the regression line and equilibrium temperatures of the first temperature sensor and the second temperature sensor respectively in advance as database, and predict the equilibrium temperature for a subject to be monitored from the database.

3. The non-invasive glucose monitor according to claim 1 characterized in that as the body surface a finger is used.

4. The non-invasive glucose monitor according to claim 1 characterized in that the heat flux measuring device is cooled with a louver or a fan.

5. The non-invasive glucose monitor according to claim 1 characterized in that the database is prepared in a form of grouping respectively with respect to items of room temperature, sex, age and anamnesis.
